(19) United States
(12) Patent Application Publication
(10) Pub. No.: US 2005/0027015 A1
(43) Pub. Date: Feb. 3, 2005

(54) Composition comprising cysteamine for improving lactation in dairy animals

(76) Inventors: Francis Chi, Hongkong (CN); Qin Tang Wen, Hongkong (CN); Tian Shui Lu, Hongkong (CN)

Correspondence Address: COLEMAN SUDOL SAPONE, P.C., 714 COLORADO AVENUE, BRIDGE PORT, CT, 06605-1601, US

(21) Appl. No.: 10/489,700

(22) PCT Filed: Sep. 18, 2002
(86) PCT No.: PCT/EP02/10518

(30) Foreign Application Priority Data
Sep. 19, 2001 (GB) 0122610.9

(51) Int. Cl. A61K 31/13

(52) U.S. Cl. 514/665

(57) ABSTRACT

The use of cysteamine, a salt thereof or a composition containing cysteamine or a salt thereof for improving lactation of lactating animals.

### BACKGROUND OF INVENTION

1. Field of Invention

The present invention relates to the uses of cysteamine or a cysteamine-containing composition for improving lactation of lactating animals, and in particular, but not limited to, dairy animals such as dairy cows. The present invention also relates to a feed and a method for improving lactation of lactating animals.

2. Description of Prior Art

Studies have indicated that growth hormones play an important role in regulating lactation in dairy animals such as diary cows. Further studies on dairy animals have shown that milk production therefrom can be increased when exogenous growth hormones are administered to the animals. However, there are a number of disadvantages in directly using growth hormones in increasing milk production. Firstly, growth hormones from different animals are seldom homogenous and different dairy animals only react to certain types of specific growth hormones. Since suitable exogenous growth hormones are normally extracted from pituitary glands, it is rather difficult and uneconomical to prepare sufficient quantity of suitable exogenous growth hormones for use on a large-scale application. Although exogenous growth hormones can now be prepared using DNA recombinant technology, exogenous growth hormones manufactured by such method are still rather expensive. Secondly, the administration of exogenous growth hormones into dairy animals is normally performed by direct injection, which is inevitably rather costly and difficult to administer in a large farm. Thirdly, it is rather difficult to control the dose administered to produce precisely the desired effect, and an overdose of exogenous growth hormones is likely to be harmful to the animals. Fourthly, residuals of these exogenous growth hormones may be passed to the dairy products and subsequently to humans through consumption thereof. Further studies in this regard are required although some scientists are concerned about the negative side effects of these exogenous growth hormones to humans.

Cysteamine is a component of co-enzyme A and works as a physiological regulator. Cysteamine has been used as an additive in feed in promoting growth of meat-producing animals. U.S. Pat. No. 4,711,897 discloses animal feed methods and feed compositions comprising cysteamine.

However, it has been identified that cysteamine is a fairly sensitive and unstable compound under normal room temperature conditions. For example, cysteamine is readily oxidized when exposed to air or at an elevated temperature. Cysteamine is highly hydroscopic. Also, cysteamine is unpalatable when taken directly by mouth. Further, ingesting cysteamine directly will cause undesirable gastro side effects. For these reasons, the use of cysteamine had for a long time been limited to direct injection of cysteamine-containing solution into meat-producing animals.

PRC Patent Publication No. CN1358499 and International Publication No. WO/0248110 disclose an improvement of a cysteamine-containing composition which can be mixed with basal animal feed to promote growth and increase in animal weight. However, there continues to exist a need for a composition, feed and/or method for increasing lactation by dairy mammals and particularly dairy cows. Preferably, the composition and method are safe and can be easily administered and inexpensive to carry out.

It is thus an object of the present invention in which the above issues are addressed, or at least to provide a useful alternative to the public.

### SUMMARY OF INVENTION

According to a first aspect of the present invention, there is provided the use of cysteamine, a salt thereof or a composition containing cysteamine or a salt thereof for improving lactation of lactating animals.

According to a second aspect of the present invention, there is provided a feed for improving lactation of lactating animals comprising a composition containing cysteamine or a salt thereof and a stabilizer.

According to a third aspect of the present invention, there is provided a method for improving lactation of lactating animals comprising steps of producing a final feed by mixing a composition containing cysteamine or a salt thereof and a stabilizer with a suitable basal feed for the animals, and feeding the animals with the final feed.

The improving lactation may be an increase in milk yields, fat-corrected milk yields, milk fat content therein and/or milk protein content therein.

Preferably, the composition comprises substantially 1 to 95 wt % cysteamine having the chemical formula of NH₂—CH₂—CH₂—SH, or a salt thereof. In particular, the composition may comprise substantially 30 wt % cysteamine.

Suitably, the composition comprises substantially 1 to 80 wt % of the stabilizer. The stabilizer is preferably selected from a group including cyclodextrin or a derivative thereof. In particular, the composition may comprise substantially 10 wt % of the stabilizer.

Advantageously, the composition further comprises ingredient(s) selected from a group including a bulking agent, a disintegration agent and a coated carrier. Preferably, the coated carrier in some embodiments is a solid carrier which is a coating soluble in intestines of the animals. The coated carrier suitably exhibits a multi-layer structure in the composition. The coated carrier is preferably adapted to remain undissolved at pH 1.5 to 3.5.

Preferably, the feed comprises substantially 400 to 1000 ppm of the composition, or substantially 1000 to 2174ppm of the composition when in its dry state. Alternatively, the feed comprises substantially 120 to 300 ppm cysteamine, or substantially 200 to 650 ppm cysteamine when in its dry state.

In some embodiments, the feed comprises other foodstuffs selected from a group including normal premix, cornmeal, cotton seed, wheat gluten, maize silage rutabaga, sugar beet pulp, apple pulp, ryegrass, fescue grass, alfalfa, feed concentrate and feed supplement.

In one embodiment, the mixing comprises directly mixing the composition with the basal feed. In another embodiment, the mixing comprises firstly preparing a premix including cysteamine or the composition, and subsequently mixing the pre-mix with the basal feed. The premix is prepared by mixing cysteamine or the composition with a food material such as cornmeal. The premix preferably comprises 5 to 25 wt % of the composition. In particular, the premix may comprise 10 to 20 wt % of the composition.

Preferably, the animals are fed with substantially 5.64 to 12.71 g cysteamine, or a salt thereof, or 19.79 to 42.36 g of the composition, per animal per day. Alternatively, the animals are fed with twice the amount of cysteamine or the composition but the frequency of the administration thereof can be reduced to every other day instead of every day. Similar results would be produced.

The lactating animals referred above may be dairy cows.

### BRIEF DESCRIPTION OF DRAWING

The invention will now be described, by way of non-limiting examples only, with reference to the accompanying drawing, in which:

FIG. 1 is a graph showing level of lactation of three groups of dairy cows in an experiment.

### DETAILED DESCRIPTION OF THE INVENTION

The present invention is based on the demonstration that cysteamine or a cysteamine-containing composition when administered to lactating animals such as diary cows has activity in improving lactation therefrom. The lactating animals or dairy animals referred hereinafter include any milk-producing animals such as dairy cows. Prior to this finding, there was no suggestion or sufficient indication that cysteamine or its variants or derivatives might have such activity. The present invention also provides a feed and a method for improving lactation of lactating animal. The use of the present invention also prolongs and heightens the milk yield during later stage of lactation of dairy animals. The invention may be practiced by directly mixing cysteamine or the cysteamine-containing composition with a suitable basal feed. Alternatively, the invention may be practiced by mixing firstly a premix made of cysteamine or the cysteamine-containing composition and other ingredients such as cornmeal, and secondly the premix with a suitable basal feed to form a final feed.

One effect of cysteamine is described in PRC Patent Publication No. CN1358499, the content of which is incorporated herein. However, a previously unknown effect of cysteamine is its effect on improving lactation of lactating animals. The effect of cysteamine or a cysteamine-containing composition on lactating animals is explained as follows. It is believed that cysteamine having a physiological activity acts as a growth stimulator. Natural cysteamine is a part of coenzyme A (also know as CoA-SH or CoA) which is a coenzyme pattern of pantothenic acid. In the course of metabolism, coenzyme A acts as the carrier of dihydrosulfuryl or variants of hydrosulfuryl which is linked with the hydrosulfuryl of coenzyme A. Experiments performed on other animals such as pigs, poultry, fowls, goats, rabbits and fish have shown that cysteamine can deplete somatostain (SS) in the animals. This increases the level of growth hormone in the blood of the animals which at the same time raises the level of various other growth stimulating factors including insulin-like growth factor I (IGF-I), insulin, triiodothyronine (T3), trthyroxine (T4) and beta-endorphin (beta-END). As regards the present invention, the growth hormone is believed to directly stimulate the development and activity of mammalian glands of dairy animals and the maintenance of lactation therefrom.

With the increase of these various growth-promoting factors, the digestive metabolic rate of the animals is correspondingly increased. It is understood that the general protein synthesis rate of the animals is accordingly increased, and thus lactation is increased.

The cysteamine-containing composition used in the present invention comprises two main ingredients of 1 to 95 wt % of cysteamine (or its salts, for example, cysteamine hydrochloride, or other pharmaceutically acceptable acid addition salts thereof) and 1 to 80 wt % of a carrier such as an inclusion compound host material. The chemical formula of cysteamine is HSCH₂CH₂NH₂. The term “cysteamine” referred hereinafter means cysteamine and/or its salt like compounds. Cysteamine and its salts are well known in the chemical literature.

The general chemical formula of a cysteamine salt is C₂H₇NS.X, where X may be HCl, H₃PO₄, bitartrate, salicylate, etc. The cysteamine used is preferably of pharmaceutically acceptable standard and the content of carbon, hydrogen, nitrogen and sulfur therein are substantially 31.14 wt %, 9.15 wt %, 18.16 wt % and 41.56 wt % respectively. While the workable content of cysteamine in the cysteamine-containing composition ranges from 1 to 95 wt %, a preferable range of 1 to 75 wt % and a more preferable range of 1 to 40 wt % of cysteamine may be used. Cysteamine is one of the main active ingredients of the cysteamine-containing composition. However, it has been identified that if the content of cysteamine in the cysteamine-containing composition exceeds 95 wt %, mixing the composition with a basal feed would be rather difficult and the effect of the composition for regulating lactation of the dairy animals would be hindered.

The inclusion compound host material may comprise mainly cyclodextrin and/or its derivatives which are selected from a group including methyl β-cyclodextrin (M-β-CD), hydropropyl β-cyclodextrin (HP-β-CD), hydroethyl β-cyclodextrin (HE-β-CD), polycyclodextrin, ethyl β-cyclodextrin (E-β-CD) and branched cyclodextrin. The general chemical formula of cyclodextrin is (C₆O₅H₉)ₙ.(C₆O₅H₉)₂ and the structural formula is as follows. 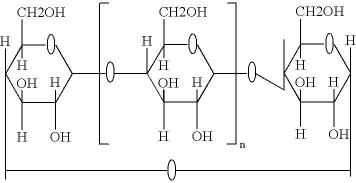
where α-CD n=4; β-CD n=5; γ-CD n=6.

(Cyclodextrin is a cyclic oligomer of alpha-D-glucopyranose.)

It is worthwhile to note that the β-CD form of cyclodextrin is preferably used because the internal diameter of its molecule is about 6-8 Å which makes it a particular suitable candidate as an inclusion compound host material for preparation of the cysteamine-containing composition, which involves the use of an inclusion process. The term “cyclodextrin” referred hereinafter means cyclodextrin and/or its derivatives. Any derivative of cyclodextrin which has the property of stabilizing and protecting cysteamine from degradation may be used. For example, any one of the group of cyclodextrin or its derivatives mentioned above may be used.

While the workable content of the inclusion compound host materials in the cysteamine-containing composition ranges from 1 to 80 wt %, a preferable workable range of 1 to 60 wt % and a more preferable workable range of 10 to 40 wt % of the inclusion compound host materials may be also be used. The actual amount of the inclusion compound host materials used will depend on the actual content of the cysteamine used in preparing the cysteamine-containing composition.

The cysteamine-containing composition may also comprise 1 to 90 wt % of fillers although a preferable workable range of 1 to 60 wt % and a more preferable workable range of 1 to 40 wt % of the fillers may also be used in the composition. The actual content will depend on the actual amount of cysteamine and inclusion compound host materials used. The fillers may be selected from a group including powdered cellulose, starch and calcium sulfate (e.g. CaSO₄.2H₂O). It is to be noted that if the content of the fillers exceeds 90 wt % in the cysteamine-containing composition, the content of the main active ingredients will thus be reduced, and the cysteamine-containing composition may become ineffective in improving lactation of the animals fed with a feed mixed therewith.

The cysteamine-containing composition may also comprise 5 to 50 wt % of disintegrants and binders although a preferable workable range of 10 to 40 wt % and a more preferable workable range of 15 to 35 wt % may also be used. The actual content will depend on the actual amount of cysteamine, the inclusion compound host material and other ingredients used. The binders and disintegrants may be selected from a group including hydropropyl starch, microbial alginate, microcrystalline cellulose and starch. It has been identified that if the content of the disintegrants and binders in the composition is less than 5 wt %, granules of the composition produced will lack the required hardness. In addition, manufacturing of the composition would become very difficult. If however the content of the disintegrants and binders is more than 50 wt %, the resulting composition will have excessive hardness, this is especially so if the content of binders represent a large portion of the mixture of the disintegrants and binders. This will result in difficult absorption of the composition by the intestines of the animals.

The cysteamine-containing composition may also comprise 0.05 to 0.3 wt % of flavoring and smelling agents which may be a flavoring essence.

The cysteamine-containing composition may also comprise 1 to 20 wt % of coating materials although a preferable workable range is 1 to 15 wt % and a more preferable workable range is 2 to 10 wt %. The actual content will depend on the actual amount of cysteamine, the inclusion compound host materials and the other ingredients used. The coating materials are preferably enteric-coated which allows dissolution in an alkaline environment such as in the intestines. The coating materials may be made of and selected from a group including cellulose acetate phthalate, starch acetate phthalate, methyl cellulose phthalate, glucose or fructose derivatives from phthalic acid, acrylic and methacrylic copolymers, polymethyl vinyl ether, partly esterified substance of maleic anhydride copolymers, lac and formogelatine. It has been identified that if the content of the coating materials is less than 1 wt %, granules of the composition may not be entirely covered by the coating materials which act as a protective layer. The cysteamine-containing composition may thus degrade before being absorbed by the intestines into the bloodstream of the animals. On the other hand, if the content of the coating materials exceeds 15 wt %, the active ingredients in the composition may not effectively be released from the composition. Thus, the intended regulation of lactation would not be achieved.

The cysteamine-containing composition used in the present invention is in the form of small granules each of which has a preferable diameter of substantially 0.28 to 0.90 mm. These granules are prepared using a micro-encapsulation method. The method involves using a macromolecular substance having inclusion property. One substance which may be used is the inclusion compound host material (which may comprise mainly cyclodextrin) is described above. The inclusion compound host material is a macromolecular substance which acts as a molecular capsule to engulf the molecules of cysteamine, whereby cysteamine in the composition is protected and insulated from light, heat, air and moisture of the surroundings. The stability of cysteamine is thus preserved. The inclusion compound host material used in the micro-encapsulation method is preferably a cyclic polysaccharide compound having 6 to 12 glucose molecules, which is produced by reacting cyclodextrin glycosidtransferase and starch in the presence of Bacillus. Various studies using acute, sub-acute and chronic toxic tests have shown that the macromolecular substance is non-toxic. Subsequent to the micro-encapsulation process, each granule may be coated with at least one and preferably a plurality of layers of the coating materials described above. The following provides a more detailed description of a method of preparing the cysteamine-containing composition used in the present invention.

In a jacketed reactor linked with polytetrafluoroethylene and equipped with a polytetrafluoroethylene coated stirrer, 4080 g of 75 wt % cysteamine hydrochloride solution in ethanol is added with mainly nitrogen being the atmosphere. The purity, melting point and burning residue of the cysteamine used are preferably 98% or above, 66 to 70° C. and 0.05% or below respectively. 1200 g β-cyclodextrin is then added into the reactor similarly under the protection of nitrogen gas. (The quality of β-cyclodextrin is in accordance with the requirements for a food additive. In particular, the dry basis purity is more than 98%; the weight loss by drying is less than 10.0%; the burning residue is less than 0.2%; the content of heavy metal is less than 10 ppm; the arsenic content is less than 2 ppm.) The mixture is then heated for 3 hours at 40° C. Heating is then stopped and stirring continues for two hours thereafter, products resulted therefrom are then grounded and sieved through a screen (e.g. 40-mesh) filter after the products have been vacuum dried at a temperature of 40-50° C. All parts of the equipment, which may come in contact with the ingredients of the composition, should preferably be made of stainless steel.

In a tank-type mixer, 4200 g (on dry basis) of the cysteamine which has undergone the inclusion process as described, 2600 g of the fillers, and 1200 g of the disintegrants and 1700 g binders are added under the protection of a dry surroundings. These ingredients are then thoroughly mixed, and a suitable amount of anhydrous ethanol may be added and then mixed therewith. The resulting mixture presents a soft material with moderate hardness, so that it can be shaped into a ball by a light hold of palms. The ball-shaped resulting mixture may then be broken up by a light touch. After the mixture is pelleted by a granulator under the protection of nitrogen, the small granules resulting therefrom is immediately introduced to a fluid-bed dryer, and is then dried at the temperature of 40-50° C. in a substantially vacuum environment.

Enteric coating materials are then prepared by a method with the following formulation: cellulose acetate phthalate 8.0 g, polyethylene glycol terephthalate 2.4 ml, ethyl acetate 33.0 ml and isopropyl acetate 33.6 ml. The resultant granules obtained above are uniformly coated under the protection of nitrogen with at least one layer but preferably a plurality of layers of the enteric coating materials described above. The enteric coating materials are dissolvable only at an alkaline environment. This can prevent the cysteamine from prematurely escaped from the composition while it is still in the stomach of the animal. Cysteamine can adversely stimulate gastric mucous of the stomach of the animals.

The resultant granules of the cysteamine-containing composition are then dried completely in a substantially vacuum dryer at a temperature of 40 to 50° C. Then, all solvents are removed. The resultant granules are then allowed to cool to room temperature, the micro-capsula were mixed with a suitable amount of flavoring and smelling agents by a cantilever double helix blender. The cysteamine-containing composition is a microcapsule with its interior having cysteamine hydrochloride and cyclodextrin, and with its exterior coated with the enteric coating materials.

The composition produced will exhibit small granular (or micro-particulate) shape having smooth surface, good flow property, and is easy to be blended with various animal feeds. The diameter of each granule of the composition is preferably 0.28 to 0.90 mm. The composition also has excellent stability. It has been found that after the composition is packaged with sealed plastic bags and stored for one year in a cool, dark and dry place, their properties remain unchanged. Therefore, they meet the requirements for a feed additive.

The composition having the particular construction described above has a number of functional advantages over cysteamine by itself. Firstly, the activity of the cysteamine contained in the composition is preserved after it has been produced. This is important as feed additive such as the composition may be stored for a relatively long period of time before use. Secondly, the composition does not cause any noticeable gastro side effects to the animals fed therewith. Thirdly, the activity of the composition is preserved not only during storage but more importantly until it reaches the intestines of the animals. Fourthly, the composition can be easily administered to farm animals on a large scale basis cost-effectively because it can be readily mixed with any basal feed. No separate procedure or injection is needed at all.

Various experiments have been conducted to demonstrate that administering a feed having cysteamine or a cysteamine-containing composition increases lactation from dairy animals, three experiments of which are described in detail as follows.

### EXPERIMENTS

### Experiment 1

Background Information

The experiment was conducted in a private dairy farm located in Guangming, PRC in April and May 2001. Thirty dairy cows were randomly selected for the experiment. Before the experiment, the cows were similar in weight, age, milk yield, and milk fat content and have similar calving number. All the cows had had approximately five months of lactation. The cows were randomly divided into three groups of ten, namely Group I, Group II and Group III, Group III being in the control. Prior to the experiment, there was no statistical difference (p>0.05) on average milk yield among the three groups of cows. Table 1 below shows data in respect of the three groups of cows and milk produced therefrom prior to the experiment.

TABLE 1
Characteristics of diary cows before experiment

| | Group | | |
|---|---|---|---|
| Characteristics | Group I | Group II | Group III |
| Number of cows | 10 | 10 | 10 |
| Average number of calving | 1 | 1 | 1 |
| Average lactation period (days) | 212.8 ± 5.20 | 208.4 ± 4.05 | 208.3 ± 3.91 |
| Average milk yield (kg/day) | 27.5 ± 4.27 | 27.4 ± 4.05 | 27.5 ± 3.97 |
| FCM (3.5%, kg/day) | 28.636 ± 5.246 | 28.773 ± 5.777 | 28.978 ± 4.973 |
| Average fat content in FCM (wt %) | 3.76 ± 0.41 | 3.78 ± 0.37 | 3.84 ± 0.39 |
| Average protein content in milk (g/day) | 856.6 ± 109.1 | 964.7 ± 159.6 | 869.8 ± 112.7 |
| Average number of somatic cells in milk | 91.7 ± 58.7 | 86.3 ± 49.99 | 84.9 ± 58.5 |
| (×1000/ml) | | | |

Note:
FCM = fat corrected milk

It is to be noted that during a lactation period, the content of milk proteins and other nutrients in milk may often remain relatively stable, whereas the fat content can vary substantially. In this regard, it has become one of the international standards to express the quantity of milk produced in terms of its equivalent quantity of fat corrected milk (FCM) for unified comparison. In the experiment, the quantity of milk produced by the three groups of dairy cows are expressed both in its original quantity (see Table 7) as well as quantity in 3.5 wt % FCM (see Table 8). It is however to be noted that there are other standards for quantifying milk production.

Materials

The recipe of two basal feeds used in the experiment are shown in Table 2 below, and the details of the respective diets of the three groups of cows are described below.

TABLE 2
Composition of basal feeds

| | For use before Apr. 30, | For use on or after Apr. 30, |
|---|---|---|
| Ingredients (kg) | 2001 | 2001 |
| Basal concentrate | 5 | 4.5 |
| Supplement feed | 3 | 3 |
| Normal premix | 1.4 | 1.3 |
| Cotton seed | 0.4 | 0.2 |
| Wheat gluten | 10 | 11 |
| Maize silage | 14 | 14.5 |
| Rutabaga | 4 | 5 |
| Sugar beet pulp | 0.5 | 0.4 |
| Apple pulp | 0.5 | 0.4 |
| Ryegrass | 4 | — |
| Fescue grass | 3 | 3 |
| Alfalfa | 0.8 | 0.5 |
| Total (Basal feed) | 46.6 | 43.8 |

Two batches of the basal feeds were prepared, the first batch was used in the first period of the experiment, i.e. from 11 Apr. to 29 Apr. 2001 while the second batch was used in the second period of the experiment, i.e. from 30 Apr. to 29 May 2001. The different basal feed was used in the second period of the experiment in order to suit the physiological requirements of the dairy cows at that particular developmental stage. As shown in Table 2 above, the first batch of feed comprised a plurality of ingredients including basal concentrate, feed supplement, normal premix, cottonseed, wheat gluten, maize silage, rutabaga, sugar beet pulp, apple pulp, ryegrass, fescue grass and alfalfa. The normal premix is formulated to compose ingredients such as vitamins and minerals, which are suitable for the cows in a particular physiological stage. The basal concentrate, supplement feed and normal premix were nutrition additives containing such as vitamins and rare elements, which were added in the feed in accordance with America's Animal National Nutrition standard. The second batch of basal feed generally comprised the same ingredients although the quantities thereof were different.

The basal feeds were used to prepare final feeds. Referring to Table 3 below, the final feeds comprised 200 g of a specific premix which was prepared by mixing a cysteamine-containing composition with a suitable food material such as cornmeal. It is to be noted that the specific premix was different from the normal premix in the basal feed.

TABLE 3
Composition of final feeds for Groups I and II
cows

| | For use before Apr. | For use on or |
|---|---|---|
| Ingredients (kg) | 30, 2001 | after Apr. 30, 2001 |
| Specific premix | 0.2 | 0.2 |
| Basal feed | 46.6 | 43.8 |
| Total (final feed) | 46.8 | 44.0 |

There were however three formulas of the specific premix, each prepared with different amounts of the cysteamine-containing composition and the food material. These formulas are illustrated as follows.
10 wt % cysteamine-containing composition/90 wt % cornmeal Formula 1:
20 wt % cysteamine-containing composition/80 wt % cornmeal Formula 2:
0 wt % cysteamine-containing composition/100 wt % cornmeal Formula 3:

While formulas 1 to 3 of the specific premix comprises up to 20 wt % of the cysteamine-containing composition, studies have shown that, in practice, the specific premix may have a content of 5 to 25 wt % of the cysteamine-containing composition and a similar effect on increasing lactation of the dairy cows will result as illustrated below.

Tables 4 to 6 below further summarize the respective diets of the Groups I to III cows during the experiment. Based on the data in Table 4, it is calculated that the concentrations of the cysteamine-containing composition in the final feeds (or diets) of the Group I cows in the first and second periods of the experiment were 427 and 455 ppm respectively. Based on the data in Table 5, it is calculated that the concentrations of the cysteamine-containing composition in the final feeds (or diets) of the Group II cows in the first and second periods were 855 and 909 ppm respectively. Referring to Table 6, no cysteamine-containing composition was added in preparing the 180 g of specific premix (Formula III). Therefore, the final feeds (or diets) of the Group III cows did not contain any cysteamine-containing composition.

TABLE 4
Diets of the Group I dairy cows

| | For Use Before | For Use On |
|---|---|---|
| Ingredients | April 30 | or After April 30 |
| Basal feed (g) | 46600 | 43800 |
| Specific premix (g) | 200 | 200 |
| Amount of cysteamine- | 10 | 10 |
| containing composition | | |
| (formula I) in specific | | |
| premix (wt %) | | |
| Total diet per day (g) | 46800 | 44000 |

TABLE 5
Diets of the Group II dairy cows

| Ingredients | Before April 30 | On or After April 30 |
|---|---|---|
| Basal feed (g) | 46600 | 43800 |
| Specific premix (g) | 200 | 200 |
| Amount of cysteamine- | 20 | 20 |
| containing composition | | |
| (formula II) in specific premix | | |
| (wt %) | | |
| Total diet per day (g) | 46800 | 44000 |

TABLE 6
Diets of the Group III (control) dairy cows

| Ingredients | Before April 30 | On or After April 30 |
|---|---|---|
| Basal feed (g) | 46600 | 43800 |
| Specific premix (g) | 180 | 180 |
| Amount of cysteamine- | 0 | 0 |
| containing composition | | |
| (formula III) in specific | | |
| premix (wt %) | | |
| Total diet per day (g) | 46780 | 43980 |

The cysteamine-containing composition being in mini-pill form comprised about 30 wt % cysteamine together with other ingredients including cyclodextrin which served as a stabilizer. The content of cyclodextrin in the composition was 10 wt %. The composition was prepared by Walcom Bio-Chemicals Industry Limited.

To form the final feeds, the appropriate formula of the specific premix was mixed with the suitable basal feed.

Studies have shown that in practice, the cysteamine-containing composition may contain 1 to 95 wt % cysteamine. In any event, it is preferred that the final feed is adjusted to contain approximately 400 to 1000 ppm of the composition. Alternatively, the final feed may be adjusted to contain approximately 120 to 300 ppm of cysteamine.

In its dry state, the feed may comprise 1000 to 2147 ppm of the composition and 200 to 650 ppm of cysteamine. The composition used in the experiment comprised 10 wt % cyclodextrin which is mentioned above. However, depending on the actual amount of cysteamine used in preparing the composition, the composition may contain 1 to 80 wt % cyclodextrin, as well as other ingredients which may include a bulking agent, a disintegration agent and a solid coated carrier. The composition is in the form of mini-pill having a multi-layer structure. The composition thus remains relatively stable at room temperature conditions and un-dissolved at a pH as low as 1.5 to 3.5 (such as in a stomach environment) after it has been ingested by the animal. However, the carrier is made of a coating material which is soluble in a higher pH environment such as in the intestines.

Procedure

All three groups of cows were kept together under the same conditions which included tether feeding and automatic drinking. The cows were allowed four hours of exercising and feeding every day. The cows were fed three times a day. During each feeding time, the cows were firstly fed with roughage and then with the respective diets (see Tables 4 to 6). The consumption of roughage was not restricted but was monitored. The cows were milked three times a day at 0600, 1400 and 2100.

During the experiment, data of milk yield expressed in its original amount and in 3.5 wt % FCM, fat content and protein content of the milk as well as the number of somatic cells in the milk were recorded and determined.

Results and Discussion

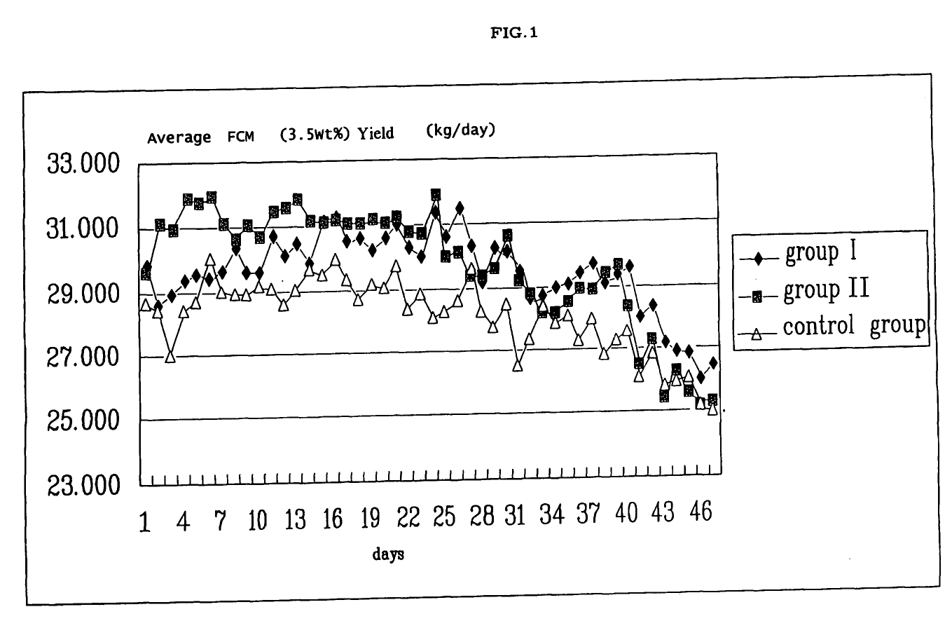

FIG. 1 shows three curves representing 3.5 wt % FCM yield. As clearly shown in the figure, the Group II cows generally had the highest 3.5 wt % FCM yield among the three groups of cows throughout the experiment. The Group I cows also had a generally higher 3.5 wt % FCM yield as compared to the Group III cows.

Table 7 below shows the data of average original milk yield of the three groups of cows. It is calculated that after feeding on the respective diets including the cysteamine-containing composition, the Groups I and II cows on average increased lactation by 4.86% and 6.88% (p<0.05) when compared to the Group III cows. Statistically, these are significant increases.

TABLE 7
Comparison of average milk yield (kg/day)

| Period | Group I | Group II | Group III |
|---|---|---|---|
| Before trial | 27.5 ± 4.27a | 27.4 ± 4.02a | 27.5 ± 4.1a |
| 1^{st} to 10^{th} day | 28.99 ± 0.43a | 29.5 ± 0.60a | 27.29 ± 0.72b |
| 11^{th} to 20^{th} day | 27.98 ± 0.37a | 28.9 ± 0.24a | 27.17 ± 0.39b |
| 21^{st} to 30^{th} day | 28.05 ± 0.59a | 28.51 ± 0.75a | 26.71 ± 0.61b |
| 31^{st} to 40^{th} day | 27.23 ± 0.77a | 27.48 ± 0.52a | 25.85 ± 0.53b |
| 1^{st} to 40^{th} day | 28.06 ± 0.84a | 28.60 ± 0.91a | 26.76 ± 0.79b |
| 41^{st} to 49^{th} day (stop | 26.09 ± 0.67a | 26.12 ± 0.76a | 25.42 ± 0.40b |
| feeding cysteamine- | | | |
| containing diet) | | | |

(Note: In each row, the figures labeled with same letters means that there is no significant difference statistically, i.e. p > 0.05; the figures labeled with different letters means that there is significant difference statistically, i.e. p < 0.05.)

For the sake of comparison, the original milk yield of the three groups cows shown in Table 7 has been converted to 3.5 wt % FCM yield which is shown in Table 8 below. The following formula is used to convert original milk yield to its corresponding FCM (3.5 wt %) yield.
Equivalent FCM=(0.44×daily milk yield)+(16×daily milk yield×fat content rate)

TABLE 8
Comparison of average FCM (3.5% wt) yield (kg/day)

| | | | Group III |
|---|---|---|---|
| Period | Group I | Group II | (control) |
| Before trial | 28.64 ± 5.25a | 28.73 ± 5.78a | 28.98 ± 4.97a |
| 1^{st} to 10^{th} day | 29.51 ± 0.49a | 31.07 ± 0.69b | 28.73 ± 0.78c |
| 11^{th} to 20^{th} day | 30.56 ± 0.41a | 31.26 ± 0.26a | 29.22 ± 0.42b |
| 21^{st} to 30^{th} day | 30.46 ± 0.67a | 30.37 ± 0.81a | 28.60 ± 0.65b |
| 31^{st} to 40^{th} day | 29.23 ± 0.34a | 28.84 ± 0.52a | 27.52 ± 0.59b |
| 1^{st} to 40^{th} day | 29.94 ± 0.63a | 30.39 ± 0.98a | 28.52 ± 0.68b |
| 41^{st} to 49^{th} day(stop | 27.10 ± 0.85a | 25.91 ± 0.78b | 25.82 ± 0.61b |
| feeding cysteamine- | | | |
| containing diet) | | | |

(Note: In each row, the figures labeled with same letters means that there is no significant difference statistically, i.e. p > 0.05; the figures labeled with different letters means that there is significant difference statistically, i.e. p < 0.05.)

It is calculated that after feeding on the respective diets including the cysteamine-containing composition, the Groups I and II cows had a 4.98% (p<0.01) and 6.56% (p<0.01) higher FCM (3.5 wt %) yield when compared to the Group III cows. Statistically, these are significant increases.

As illustrated above, the Groups I and II cows significantly improved their lactation in terms of its original milk yield and 3.5 wt % FCM yield after feeding on cysteamine-containing diets.

It is to be noted that after each calving, the lactation of a cow normally lasts for approximately 300 days. The above experimental data indicates that administering cysteamine/cysteamine-containing composition to dairy cows at least heightens lactation. It appears that cysteamine can also prolong lactation since when the lactation is maintained at a higher level for a longer time, the lactation should be prolonged to a certain extent, as shown by the curve representing the milk yield of the Group I cows in FIG. 1.

Table 9 below shows the data of average fat content in the milk produced by the three groups of cows.

TABLE 9
Comparison of average fat content (wt %) in milk

| | | | Group III |
|---|---|---|---|
| Period | Group I | Group II | (control) |
| Before trial | 3.76 ± 0.41a | 3.78 ± 0.37a | 3.84 ± 0.40b |
| 1^{st} to 10^{th} day | 3.62 ± 0.39a | 3.86 ± 0.26b | 3.84 ± 0.26b |
| 11^{th} to 20^{th} day | 4.10 ± 0.42a | 4.00 ± 0.24a | 3.99 ± 0.23a |
| 21^{st} to 30^{th} day | 4.06 ± 0.42a | 3.90 ± 0.21b | 3.95 ± 0.16b |
| 31^{st} to 40^{th} day | 4.02 ± 0.45a | 3.80 ± 0.22b | 3.91 ± 0.20c |
| 1^{st} to 40^{th} day | 3.95 ± 0.45a | 3.89 ± 0.23a | 3.92 ± 0.21a |
| 41^{st} to 49^{th} day (stop | 3.69 ± 0.53a | 3.53 ± 0.20a | 3.68 ± 0.24a |
| feeding cysteamine- | | | |
| containing diet) | | | |

(Note: In each row, the figures labeled with same letters means that there is no significant difference statistically, i.e. p > 0.05; the figures labeled with different letters means that there is significant difference statistically, i.e. p < 0.05.)

Before the experiment, there is no significant difference of milk fat content among the three groups of cows (p>0.05, see Table 1). It is calculated that after feeding on the respective diets including the cysteamine-containing composition, the milk produced by the Group I cows had an average of 0.77% (p<0.05) higher fat content than that of the Group III cows. It is also calculated that after feeding on the respective diets including the cysteamine-containing composition, the milk produced by the Group II cows had an average of 0.77% (p<0.5) lower fat content than that of the Group III cows during the experiment, although the difference is not significant statistically. As illustrated above, there is no significant difference in fat content in milk produced by the Groups I and II cows after their diets were added with cysteamine/cysteamine-containing composition. This indicates that the quality of milk produced by cows feeding on a cysteamine-containing diet can at least be maintained if not increased.

It is however to be noted that during the first ten-day period of the experiment, the Group I cows appeared to have reacted more slowly to its cysteamine-containing diet in terms of the fat content in its milk in comparison to the Group II cows. However, the fat content in the milk produced by the Group I cows had risen rapidly and peaked at 4.10 wt % during the period of 11^{st} to 20^{th} day of the experiment. To the contrary, the Group II cows appeared to have reacted relatively more rapidly to its cysteamine-containing diet in the first ten-day period. In particular, the fat content was 3.78 wt % before the experiment but rose to 3.86 wt % in the first ten-day period of the experiment, and the fat content peaked at 4.00 wt %. It is to be noted that the milk produced by the Group II cows had the lowest fat content among the three groups when all the cows were stopped feeding on the respective cysteamine-containing diets. It is thus indicated in the experiment that cysteamine or a cysteamine-containing composition when administered at an appropriate dose and/or a particular lactation stage can increase the content of milk fat in the milk.

Table 10 below shows the data of average milk protein content in milk produced by the three groups of cows.

TABLE 10
Comparison of average milk protein content
(g/cow/day)

| | | | Group III |
|---|---|---|---|
| Period | Group I | Group II | (control) |
| Before trial | 856.6 ± 109a | 864.7 ± 159.6a | 869.8 ± 112.7a |
| 1^{st} to 10^{th} day | 926.9 ± 89.4a | 941.8 ± 109.2a | 904.7 ± 116.3b |
| 11^{th} to 20^{th} day | 911.9 ± 80.1a | 917.6 ± 126.8a | 875.8 ± 82.7b |
| 21^{st} to 30^{th} day | 928.3 ± 75.8a | 903.4 ± 120.7b | 878.2 ± 106.9c |
| 31^{st} to 40^{th} day | 912.8 ± 69.5a | 868.7 ± 121.3b | 875.2 ± 103.2b |
| 1^{st} to 40^{th} day | 920.1 ± 28.8a | 907.9 ± 30.6a | 883.5 ± 24.2b |
| 41^{st} to 49^{th} day (stop | 877.1 ± 79.4a | 830.9 ± 116.5b | 850.7 ± 89.0c |
| feeding cysteamine- | | | |
| containing diet) | | | |

(Note: In each row, the figures labeled with same letters means that there is no significant difference statistically, i.e. p > 0.05; the figures labeled with different letters means that there is significant difference statistically, i.e. p < 0.05.)

Before the experiment, the protein content in milk (g/day/cow) produced by the Group III cows was about 869.8 g which was slightly higher than that of the Groups I and II cows, although the difference was not significant statistically. After the Groups I and II cows were fed with the respective cysteamine-containing diets, the protein content in their milk increased significantly by 4.14% and 2.76% respectively (p<0.05) as compared to the Group III cows. After the Groups I and II cows stopped feeding on the respective cysteamine-containing diets, the protein content reduced close to the pre-experiment levels. It is therefore illustrated that feeding on cysteamine-containing diet not only increases original milk yield, FCM yield and milk fat content but also milk protein content.

Table 11 below shows the data of the number of somatic cells in the milk produced by the three groups of cows.

TABLE 11
Comparison of somatic cells (×10³/ml) in milk

| Period | Group I | Group II | Group III (control) |
|---|---|---|---|
| Before trial | 91.7 ± 64.9a | 86.3 ± 55.3a | 84.9 ± 64.7a |
| 1^{st} to 10^{th} day | 96.6 ± 92.8a | 102.0 ± 78.0a | 116.9 ± 129.0a |
| 11^{th} to 20^{th} day | 90.0 ± 82.9a | 87.2 ± 64.9a | 59.9 ± 33.0b |
| 21^{st} to 30^{th} day | 122.8 ± 113.3a | 89.8 ± 60.9b | 69.5 ± 42.7b |
| 31^{st} to 40^{th} day | 155.2 ± 155.1a | 92.1 ± 58.4b | 79.0 ± 59.7b |
| 1^{st} to 40^{th} day | 116.1 ± 113.0a | 92.8 ± 63.6b | 81.3 ± 76.3b |
| 41^{st} to 49^{th} day | 115.2 ± 115.9a | 96.2 ± 54.5b | 93.9 ± 45.5b |

(Note:
In each row, the figures labeled with same letters means that there is no significant difference statistically, i.e. p > 0.05; the figures labeled with different letters means that there is significant difference statistically, i.e. p < 0.05.)

After feeding on the respective cysteamine-containing diets, the milk produced by the Groups I and II cows had a higher number of somatic cells. However, the difference remained at an acceptable safety level.

In the experiment, Formula II of the specific premix comprised twice as much the cysteamine-containing composition. In other words, the Group II cows were fed with twice as much cysteamine/cysteamine-containing compound as compared to the Group I cows. While the milk yield did increase as compared to that of the Group III cows, the increase was not twice as much as compared to the Group I cows. It is believed there is a certain physiological limit a cow can react to cysteamine. In the event that a relatively high amount of cysteamine were taken by a cow, the physiology would still not react exceeding its limit. This is desirable as the dosage of the cysteamine-containing composition in the diet is not critical in that an overdose thereof is not dangerous to the animal. Thus, administering the cysteamine-containing composition in dairy cows is safe and easy to carry out.

It is to be noted that a final feed has a relatively high water content (e.g. 10 to 60 wt % water content) and the precise concentrations of cysteamine and cysteamine-containing composition may thus vary depending on the water content thereof and the humidity of the surroundings. In this regard, it is worthwhile to express a workable range of the concentration of cysteamine and cysteamine-containing composition in the final feed based on its dry weight (state). For example, studies have determined that the concentration of 1000 to 2174 ppm of the cysteamine-containing composition used in the above examples in the final feed based on its dry weight will produce the similar results in increasing lactation as shown in the examples. Similarly, it is calculated that the concentration of 200 to 650 ppm of cysteamine in the final feed based on its dry weight will likewise produce similar results.

It is also to be noted that while the administration of cysteamine and the cysteamine-containing composition to the diary cows are performed via its diet, it is envisaged that cysteamine and the cysteamine-containing composition may Is likewise be fed to the dairy cows in the form of pills, tablets or other suitable state separately or together with a food material and this would produce the same effect. Studies have shown that the effective amount of cysteamine administered to raise the dairy cows may be substantially 5.64 to 12.71 g per cow per day. The effective amount of cysteamine-containing composition may be substantially 18.79 to 42.36 g per cow per day.

### Experiment 2

Background Information

100 black & white dairy cows were used in the experiment. Before the experiment, the average body weight of the dairy cows was about 600 kg; the average lactation period of the cows was about 135^{th} day and the average milk production was about 35 kg. The quality of the milk produced by the cows prior to the experiment was similar. The cows were not administered with any cysteamine products beforehand.

The cows were equally divided into one test group and one respective control group. Each of the test and control groups are further divided into four sub-groups during their 19^{th} week of lactation, namely Groups I to IV, according to their actual milk production, calving history and stage of lactation. The prior milk production (MP) of the Groups I to IV cows were MP≦30 kg, 30 kg<MP≦35 kg, 35 kg<MP≦40 kg, 40 kg<MP respectively.

Procedure and Materials

The experiment was carried out during the 20^{th} to 32^{nd} week of lactation of the cows. The actual experiment was preceded by a two-day adaptation period. At different periods during the experiment, the test sub-groups of cows were administered with different amounts of a cysteamine-containing composition via a basal feed. The composition is identical to that used in Experiment 1. In particular, the composition comprises substantially 30% wt cysteamine.

The control group of cows was fed with the same basal feed as the test group of cows. The amount of basal feed used was adjusted according to the actual milk production at the time.

All the cows were fed three times daily. During each feeding time, the cows were fed firstly with roughage and then the basal feed mixed with or without the composition. The cows were milked three times daily at 0730, 1430 and 2130.

The milk collected was measured for its quantity and analyzed for its milk fat content and milk protein content.

Results and Discussion

Tables 12 to 16 below summarize the experimental data of Experiment 2.

TABLE 12
Content of milk fat (wt %) produced by the test and control groups of cows

| | CCC | | | | | | | | | |
|---|---|---|---|---|---|---|---|---|---|---|
| | 20 | | 30 | | 40 | | 60 | | mean | |
| Sub- | Group | | | | | | | | | |
| group | T | C | T | C | T | C | T | C | T | C |
| I | 3.67 ± 0.70 | 3.77 ± 0.65 | 3.66 ± 0.79 | 3.91 ± 0.68 | 3.63 ± | 3.82 ± 0.57 | 3.52 ± 0.43 | 3.45 ± 0.77 | 3.69 ± 0.09* | 3.73 ± 0.20 |
| | | | | | 0.72 | | | | | |
| II | 3.95 ± 0.41 | 3.81 ± 0.49 | 3.87 ± 0.45 | 3.74 ± 0.60 | 3.98 ± | 3.78 ± 0.47 | 3.62 ± 0.45 | 3.45 ± 0.77 | 3.89 ± 0.15* | 3.73 ± 0.16 |
| | | | | | 0.43* | | | | | |
| III | 3.58 ± 0.38 | 3.62 ± 0.58 | 3.51 ± 0.56 | 3.54 ± 0.66 | 3.73 ± | 3.86 ± 0.53 | 3.65 ± 0.47 | 3.64 ± 0.33 | 3.64 ± 0.15 | 3.70 ± 0.17 |
| | | | | | 0.46 | | | | | |
| IV | 3.95 ± 0.38^{#} | 3.62 ± 0.37 | 3.82 ± 0.38* | 3.54 ± 0.46 | 3.92 ± | 3.48 ± 0.55 | 3.89 ± 0.43* | 3.37 ± 0.41 | 3.89 ± 0.12** | 3.50 ± 0.12 |
| | | | | | 0.48** | | | | | |
| | mean | | mean | | mean | | mean | | mean | |
| | 3.80 ± 0.49 | 3.72 ± 0.51 | 3.73 ± 0.57 | 3.67 ± 0.61 | 3.84 ± | 3.75 ± 0.53 | 3.65 ± 0.45^{#} | 3.50 ± 0.47 | 3.77 ± 0.11 | 3.66 ± 0.12 |
| | | | | | 0.53 | | | | | |

(Note: CCC = Cysteamine-containing composition, g per cow per day (g/c/d); T = Test group; C = Control group; *p < 0.05; **p < 0.01; ^{#}0.05 < p < 0.15)

Referring to Table 12, it is shown that after administered with a feed containing the cysteamine-containing composition during the experiment, there was an average increase of milk fat content in the milk produced by the test cows by about 3.01% (=[3.77 wt %−3.66 wt %]/3.66 wt %×100%). In the test Group II of cows, the average increase of milk fat content was about 4.29% (=[3.89 wt %−3.73 wt %]/3.73 wt %×100%). In the test Group IV of cows, the average increase of milk fat content was more significant by about 11.14% (=[3.89 wt %−3.50 wt %]/3.50 wt %×100%). Since the Group IV of cows were the cows with the highest milk yield before the experiment, it is shown that the effect of cysteamine on milk fat content is more prominent on the dairy animals with already higher milk production.

Referring to the four test sub-groups of cows administered with 20 g/c/d, 30 g/c/d, 40 g/c/d and 60 g/c/d of the composition respectively, it is calculated that the increase in milk fat content in the milk produced are 2.15%, 1.63%, 2.40% and 4.29% respectively.

TABLE 13
Content of milk protein (wt %) produced by the test and control groups of cows

| CCC | | | | |
|---|---|---|---|---|
| 20 | 30 | 40 | 60 | mean |

| Sub- | Group | | | | | | | | | |
|---|---|---|---|---|---|---|---|---|---|---|
| group | T | C | T | C | T | C | T | C | T | C |

| I | 3.10 ± | 3.41 ± 0.33 | 3.06 ± | 3.32 ± 0.36 | 2.96 ± 0.17* | 3.22 ± 0.35 | 2.99 ± 0.15 | 2.73 ± 0.81 | 3.01 ± 0.06* | 3.21 ± 0.05 |
|---|---|---|---|---|---|---|---|---|---|---|
| | 2.33^{#} | | | | | | | | | |
| II | 3.12 ± | 2.91 ± 0.22 | 3.14 ± | 2.94 ± 0.24 | 3.00 ± 0.25^{#} | 2.93 ± 0.21 | 3.00 ± 0.21 | 3.01 ± 0.22 | 3.06 ± 0.08** | 2.94 ± 0.04 |
| | 0.28* | | 0.21** | | | | | | | |
| III | 2.96 ± | 2.94 ± 0.25 | 3.01 ± | 2.98 ± 0.34 | 2.91 ± 0.25 | 2.88 ± 0.27 | 2.92 ± 0.21 | 2.89 ± 0.22 | 2.95 ± 0.06 | 2.92 ± 0.05 |
| | 0.28 | | 0.34 | | | | | | | |
| IV | 2.84 ± | 2.63 ± 0.13 | 2.92 ± | 2.67 ± 0.16 | 2.86 ± 0.26** | 2.66 ± 0.19 | 2.88 ± 0.27 | 2.76 ± 0.21 | 2.88 ± 0.05** | 2.67 ± 0.06 |
| | 0.27* | | 0.26** | | | | | | | |
| | mean | | mean | | mean | | mean | | mean | |
| | 3.03 ± | 2.92 ± 0.03 | 3.00 ± | 2.92 ± 0.31 | 2.92 ± 0.24 | 2.90 ± 0.28 | 2.96 ± 0.21^{#} | 2.90 ± 0.33 | 3.06 ± 0.17* | 2.92 ± 0.04 |
| | 2.08* | | 0.29^{#} | | | | | | | |

(Note: CCC = Cysteamine-containing composition, g per cow per day (g/c/d); T = Test group; C = Control group; *p < 0.05; **p < 0.01; ^{#}0.05 < p < 0.15)

Table 13 shows that the average increase of the milk protein content in milk produced by the test cows was about 4.79% (=[3.06 wt %−2.92 wt %]/2.92 wt %×100%). In particular, the increase of the milk protein content in milk produced by the test Group II of cows was about 4.08% (=[3.06 wt %−2.94 wt %]/2.94 wt %×100%). The increase of the milk protein content in milk produced by the test Group IV of cows was more significant by about 7.87% (=[2.88 wt %−2.67 wt %]/2.67 wt %×100%). Since the Group IV cows were the cows with the highest yield of milk production before the experiment, it is shown that the effect of cysteamine on milk protein content is more prominent on the dairy animals with already higher milk production.

Referring to the dosage effects of the composition on milk protein content, the dose of 20 g/c/d increased the milk protein content significantly in the test sub-groups of cows by about 3.77% (=[3.03 wt %−2.92 wt %]/2.92 wt %×100%).

TABLE 14
Production of 3.5 wt % FCM milk by the test and control groups of cows

| | CCC | | | | | | | | | |
|---|---|---|---|---|---|---|---|---|---|---|
| | 20 | | 30 | | 40 | | 60 | | mean | |
| | Group | | | | | | | | | |
| Subgroup | T | C | T | C | T | C | T | C | T | C |
| I | 29.4 ± 3.4 | 28.3 ± 3.7 | 28.9 ± 4.4 | 25.4 ± 5.0 | 25.9 ± 4.5 | 22.4 ± 6.2 | 22.6 ± 3.3 | 19.9 ± 7.0 | 26.7 ± 3.2 | 24.0 ± 5.2 |
| II | 34.9 ± 3.4 | 35.2 ± 3.1 | 33.4 ± 4.7 | 33.0 ± 4.0 | 30.9 ± 5.0 | 29.9 ± 4.6 | 26.6 ± 5.8 | 26.0 ± 5.2 | 31.0 ± 3.2 | 31.4 ± 3.4 |
| III | 37.8 ± 3.1 | 37.8 ± 4.6 | 35.5 ± 4.3 | 35.5 ± 5.9 | 33.7 ± 5.6 | 34.1 ± 5.9 | 29.7 ± 5.5 | 29.4 ± 6.1 | 34.2 ± 4.0 | 34.2 ± 4.7 |
| IV | 45.0 ± 4.8 | 43.6 ± 4.6 | 42.1 ± 2.0 | 40.5 ± 6.5 | 38.7 ± 3.4^{#} | 34.2 ± 7.3 | 35.0 ± 2.0^{#} | 30.9 ± 5.9 | 40.2 ± 12.0 | 37.3 ± 5.8 |
| | mean | | mean | | Mean | | mean | | mean | |
| | 36.1 ± | 36.7 ± 5.6 | 34.4 ± 5.8 | 34.2 ± 6.4 | 31.8 ± 6.2 | 31.0 ± 6.6 | 27.9 ± 6.2 | 27.1 ± 6.4 | 32.4 ± 5.6 | 32.3 ± 5.7 |
| | 25.8 | | | | | | | | | |

(Note: CCC = Cysteamine-containing composition, g per cow per day (g/c/d); T = Test group; C = Control group; ^{#}0.05 < p < 0.15)

Table 14 shows that the test Groups I and IV of cows increased their 3.5 wt % FCM milk yield very significantly by 11.25% (=[26.7 kg/day−24.0 kg/day]/24.0 kg/day×100%) and 7.7% (=[40.2 kg/day−37.3 kg/day]/37.3 kg/day×100%) respectively, while in the test Groups II and III of cows, the production of 3.5 wt % FCM was not altered significantly as compared to that of the respective control groups. Since the test Group I of cows were the cows with the lowest milk yield before the experiment, it is shown that the positive effect of cysteamine on 3.5% FCM yield is more prominent on dairy animals with lower FCM yield.

TABLE 15
Production of original milk (kg/day) by the test and control groups of cows

| | CCC | | | | | | | | | |
|---|---|---|---|---|---|---|---|---|---|---|
| | 20 | | 30 | | 40 | | 60 | | Mean | |
| | Group | | | | | | | | | |
| Subgroup | T | C | T | C | T | C | T | C | T | C |
| I | 28.4 ± | 25.8 ± 1.3 | 28.1 ± 0.6* | 23.7 ± 1.3 | 25.2 ± 0.8^{#} | 21.1 ± 1.9 | 22.0 ± 0.8* | 19.7 ± 2.1 | 25.9 ± 0.7** | 23.1 ± 0.9 |
| | 0.5^{#} | | | | | | | | | |
| II | 32.2 ± | 33.6 ± 0.5 | 31.2 ± 0.7 | 31.7 ± 0.5 | 28.6 ± 0.8 | 28.6 ± 0.8 | 25.9 ± 1.0 | 26.0 ± 0.9 | 29.6 ± 0.5 | 29.9 ± 0.6 |
| | 0.6^{#} | | | | | | | | | |
| III | 37.2 ± | 36.8 ± 1.0 | 35.4 ± 0.7 | 35.2 ± 1.2 | 32.4 ± 1.1 | 32.2 ± 1.4 | 28.9 ± 1.2 | 28.6 ± 1.4 | 33.5 ± 0.7 | 33.3 ± 0.7 |
| | 0.6 | | | | | | | | | |
| IV | 42.0 ± | 43.0 ± 0.7 | 40.1 ± 1.0 | 40.2 ± 1.9 | 36.3 ± 1.3 | 34.0 ± 1.9 | 33.1 ± 1.2 | 31.3 ± 1.6 | 37.9 ± 1.0^{#} | 36.8 ± 1.4 |
| | 0.7 | | | | | | | | | |
| | mean | | Mean | | Mean | | Mean | | Mean | |
| | 34.2 ± | 35.2 ± 0.8 | 33.0 ± 0.7 | 33.7 ± 0.8 | 30.1 ± 0.7 | 29.6 ± 0.8 | 27.1 ± 0.7 | 26.9 ± 0.8 | 31.1 ± 0.3 | 31.0 ± 0.4 |
| | 0.7 | | | | | | | | | |

(Note: CCC = Cysteamine-containing composition, g per cow per day (g/c/d); T = Test group; C = Control group; *p < 0.05; **p < 0.01; ^{#}0.05 < p < 0.15)

TABLE 16
Production of milk protein (kg/day) by the test and control groups of cows

| | CCC | | | | | | | | | |
|---|---|---|---|---|---|---|---|---|---|---|
| | 20 | | 30 | | 40 | | 60 | | mean | |
| Sub- | Group | | | | | | | | | |
| group | T | C | T | C | T | C | T | C | T | C |
| I | 0.88 ± 0.08 | 0.88 ± 0.17 | 0.86 ± 0.09 | 0.79 ± 0.16 | 0.74 ± 0.06 | 0.69 ± 0.19 | 0.67 ± 0.06 | 0.56 ± 0.27 | 0.79 ± 0.12^{#} | 0.71 ± 0.18 |
| II | 1.00 ± 0.11 | 0.97 ± 0.10 | 0.93 ± 0.25 | 0.93 ± 0.10 | 0.86 ± 0.13 | 0.83 ± 0.10 | 0.77 ± 0.13 | 0.78 ± 0.12 | 0.89 ± 0.12 | 0.88 ± 0.08 |
| III | 1.10 ± 0.12 | 1.08 ± 0.14 | 1.06 ± 0.14 | 1.05 ± 0.16 | 0.94 ± 0.14 | 0.93 ± 0.17 | 0.84 ± 0.13 | 0.83 ± 0.17 | 0.99 ± 0.11 | 0.97 ± 0.14 |
| IV | 1.19 ± 0.09^{#} | 1.13 ± 0.07 | 1.17 ± 0.08^{#} | 1.07 ± 0.12 | 1.04 ± 0.09* | 0.90 ± 0.10 | 0.95 ± 0.07^{#} | 0.86 ± 0.10 | 1.09 ± 0.11 | 0.99 ± 0.14 |
| | mean | | Mean | | Mean | | Mean | | Mean | |
| | 1.03 ± 0.15 | 1.02 ± 0.14 | 1.01 ± 0.15 | 0.98 ± 0.15 | 0.89 ± 0.15 | 0.85 ± 0.15 | 0.78 ± 0.17 | 0.80 ± 0.14 | 0.93 ± 0.15 | 0.91 ± 0.14 |

(Note: CCC = Cysteamine-containing composition, g per cow per day (g/c/d); T = Test group; C = Control group; ^{#}0. 05 < p < 0.15)

Table 15 shows that the original milk yields of the test Group I of cows increased very significantly by 12.12% (=[25.9 kg/day−23.1 kg/day]/23.1 kg/day×100%).

Table 16 shows that the actual quantity of milk protein produced by the test Group I of cows increased by 11.27% (=[0.79 kg/day−0.71 kg/day]/0.71 kg/day×100%). However, the data in Tables 12 to 14 is more representative in illustrating the positive effects of cysteamine on improving lactation of dairy animals.

TABLE 17
Effect of the composition on feed to milk ratio

| | CCC | | | | | | | | | |
|---|---|---|---|---|---|---|---|---|---|---|
| | 20 | | 30 | | 40 | | 60 | | mean | |
| Sub- | Group | | | | | | | | | |
| group | T | C | T | C | T | C | T | C | T | C |
| I | 0.52 ± | 0.56 ± 0.03 | 0.54 ± 0.09 | 0.60 ± 0.08 | 0.54 ± 0.06* | 0.68 ± 0.10 | 0.58 ± 0.08 | 0.68 ± 0.14 | 0.54 ± 0.06** | 0.63 ± 0.11 |
| | 0.01 | | | | | | | | | |
| II | 0.46 ± | 0.44 ± 0.03 | 0.47 ± 0.04 | 0.46 ± 0.04 | 0.50 ± 0.05 | 0.50 ± 0.06 | 0.51 ± 0.08 | 0.50 ± 0.09 | 0.48 ± 0.06 | 0.48 ± 0.06 |
| | 0.04* | | | | | | | | | |
| III | 0.39 ± | 0.40 ± 0.03 | 0.41 ± 0.03 | 0.42 ± 0.05 | 0.42 ± 0.06 | 0.43 ± 0.06 | 0.46 ± 0.08 | 0.47 ± 0.10 | 0.41 ± 0.04 | 0.43 ± 0.05 |
| | 0.02 | | | | | | | | | |
| IV | 0.35 ± | 0.34 ± 0.02 | 0.36 ± 0.03 | 0.37 ± 0.05 | 0.37 ± 0.03 | 0.40 ± 0.06 | 0.40 ± 0.04 | 0.42 ± 0.05 | 0.38 ± 0.02 | 0.39 ± 0.04 |
| | 0.02 | | | | | | | | | |
| | mean | | mean | | mean | | mean | | mean | |
| | 0.45 ± | 0.43 ± 0.06 | 0.45 ± 0.08 | 0.45 ± 0.08 | 0.47 ± 0.07 | 0.49 ± 0.10 | 0.49 ± 0.09 | 0.50 ± 0.12 | 0.46 ± 0.07 | 0.47 ± 0.09 |
| | 0.07 | | | | | | | | | |

(Note: CCC = Cysteamine-containing composition, g per cow per day (g/c/d); T = Test group; C = Control group)

Table 17 above shows that the feed to milk ratio is generally not affected whether or not the cows are administered with the composition. This is important from a cost point of view.

### Experiment 3

Background Information

Thirty-two black & white dairy cows were used in the experiment and were randomly divided into four groups, namely test Groups I to III, and a control group. Each group had eight cows. The characteristics of milk produced by the cows before the experiment is summarized in Table 17 below.

TABLE 18
Dairy performance of the four groups of cows
before the experiment

| | Group | | | |
|---|---|---|---|---|
| Characteristics | I | II | III | Control |
| Number of cows | 8 | 8 | 8 | 8 |
| Calving number | 1 | 1 | 1 | 1 |
| Day of lactation | 20 | 20 | 20 | 20 |
| Daily milk production, | 26.90 ± 1.91 | 26.84 ± 4.51 | 26.76 ± 3.77 | 26.74 ± 4.47 |
| kg/cow/day | | | | |
| 3.5 wt % FCM, kg/cow/day | 28.22 ± 2.37 | 29.81 ± 2.48 | 28.35 ± 3.99 | 29.73 ± 3.35 |
| Milk fat content, wt % | 3.90 ± 0.56 | 4.19 ± 0.58 | 3.87 ± 0.93 | 4.20 ± 0.78 |
| Daily milk protein | 698.88 ± 63.64 | 754.59 ± 49.45 | 744.93 ± 52.86 | 713.29 ± 76.97 |
| production, g/cow/day | | | | |
| Number of somatic cells in | 1097.4 ± 1070.2 | 803.00 ± 1113.4 | 126.90 ± 87.10 | 99.90 ± 136.50 |
| milk, 10³/ml | | | | |

Procedure and Materials

The cysteamine-containing composition used was the same composition in Experiments 1 and 2 comprising substantially 30 wt % cysteamine.

There were three stages in the experiment. In the first stage of the experiment, the feed for the test Groups I to III of cows was mixed with the composition such that each cow in the three test groups was fed with 20 g, 30 g and 40 g of the composition respectively daily. No cysteamine-containing composition was mixed in the feed for the control group of cows. The first stage of the experiment lasted for thirty-seven days and was preceded by a three-day adaptation period.

During the second stage of the experiment, the composition was not added to the feed for the three test groups of cows. However, each cow in the control group was fed with 20 g of the composition daily via its feed. The second stage of the experiment lasted for seven days.

During the third stage of the experiment, the three test groups of cows were fed with the feed mixed with the composition such that 20 g of the composition was taken by each cow daily. Each cow in the control group was fed with 40 g of the composition every other day via the feed. The third stage of the experiment lasted for seven days.

The daily milk yield, milk fat content, milk protein content and somatic cells in milk were measured and recorded. The cows were fed three times a day and milked also three times a day.

Results and Discussion

Tables 19 to 24 summarize the data from the experiment.

TABLE 19
Effect of the composition on original milk yield
(kg/cow/day)

| | Group | | | |
|---|---|---|---|---|
| Time | Test Group I | Test Group II | Test Group III | Control Group |
| Before experiment | 26.90 ± 1.91^{a} | 26.84 ± 4.51^{ab} | 26.76 ± 3.77^{abc} | 26.74 ± 4.47^{abcd} |
| Stage one | | | | |
| 1^{st} to 10^{th} day | 27.52 ± 0.52^{a} | 28.05 ± 0.20^{b} | 27.40 ± 0.56^{ac} | 27.89 ± 0.44^{abcd} |
| 11^{th} to 20^{th} day | 28.87 ± 0.34^{a} | 26.39 ± 0.91^{B} | 26.59 ± 0.26^{bC} | 27.61 ± 0.59^{bd} |
| 21th to 30^{th} day | 28.45 ± 0.53^{a} | 26.77 ± 0.92^{B} | 26.57 ± 0.23^{bc} | 26.49 ± 0.20^{bcD} |
| 31st to 37^{th} day | 27.64 ± 0.84^{a} | 27.24 ± 0.42^{ab} | 26.09 ± 0.20^{ac} | 27.08 ± 1.12^{abcd} |
| Average: 1^{st} to | 28.14 ± 0.74^{A} | 27.15 ± 0.91^{B} | 26.76 ± 0.58^{BC} | 27.27 ± 0.76^{BCD} |
| 37^{th} day | | | | |
| Stage two | 26.95 ± 0.52^{a} | 25.94 ± 1.07^{ab} | 25.44 ± 0.13^{bC} | 25.09 ± 0.64^{bcd} |

| | | |
|---|---|---|
| Stage three | 25.51 ± 0.32^{a} | 27.00 ± 0.42^{B} |

(Note:
In the table, same letters in a row means that the difference of their values is insignificant, i.e. p > 0.05, different letters in a same row means that the difference of their values is significant p < 0.05; different letter size in a column means their values are significant, i.e. p < 0.01)

Table 19 above shows that the test Group I of cows increased their original milk yield by 7.41% (=[26.95−25.09]/25.09×100%) when compared to that of the cows in the control group. In particular, the increase in milk yield was more significant after the first ten-day period. For instance, the increase in milk yield of the test Group I of cows during the 11^{th} to 20^{th} day and 21^{st} to 30^{th} day was 4.56% (=[28.87−27.61]/27.61×100%) and 7.40% (=[28.45−26.49]/26.49×100%) respectively. It is thus illustrated that administering an appropriate dose of the composition to dairy animals is effective in increasing their original milk yield. In particular, the increase is more noticeable after an initial short period of the administration of the composition.

TABLE 20
Effect of the composition on quantity of milk fat
(kg/cow/day) in milk

| | Group | | | |
|---|---|---|---|---|
| | | | | Control |
| Time | Test Group I | Test Group II | Test Group III | Group |
| Before the | 3.90 ± 0.56^{a} | 4.19 ± 0.58^{ab} | 3.87 ± 0.93^{abc} | 4.20 ± |
| experiment | | | | 0.78^{abcd} |
| Stage one | | | | |
| 1^{st} to 20^{th} day | 3.63 ± 0.68^{a} | 3.63 ± 0.61^{ab} | 4.07 ± 0.54^{abc} | 3.63 ± |
| | | | | 0.44^{abd} |
| 21^{st} to 37^{th} day | 4.26 ± 0.59^{a} | 4.35 ± 0.78^{ab} | 4.36 ± 0.76^{abc} | 4.17 ± |
| | | | | 0.54^{abcd} |
| Average: 1^{st} to | 3.95 ± 0.70^{a} | 3.99 ± 0.77^{ab} | 4.21 ± 0.65^{abc} | 3.90 ± |
| 37^{th} day | | | | 0.55^{abcd} |
| Stage two | 3.69 ± 0.53^{a} | 3.69 ± 0.30^{ab} | 3.73 ± 0.82^{abc} | 3.58 ± |
| | | | | 0.35^{abcd} |

| | | |
|---|---|---|
| Stage three | 3.94 ± 0.31^{a} | 3.98 ± |
| | | 0.48^{ab78} |

(Note:
In the table, same letters in a row means that the difference of their values is insignificant, i.e. p > 0.05, different letters in a same row means that the difference of their values is significant p < 0.05; different letter size in a column means their values are significant, i.e. p < 0.01)

Table 20 above shows that the milk fat in milk produced by the test Group III of cows increased by 7.95% (=[4.21−3.90]/3.90×100%) in stage one of the experiment. In particular, the increase in milk fat was particularly significant during the first twenty-day period of the experiment, i.e. 12.12% (=[4.07−3.63]/3.63×100%).

In stage two of the experiment, the test groups of cows were stopped feeding on the cysteamine-containing diet. However, the quantity of milk fat in the milk produced thereby was still higher when compared to that of the control group of cows. This illustrates that the effect of cysteamine lasted for some time after the administration of the composition was stopped.

TABLE 21
Effect of the composition on 3.5 wt % FCM yield,
kg/cow/day

| | Group | | | |
|---|---|---|---|---|
| Time | Test Group I | Test Group II | Test Group III | Control Group |
| Before | 28.22 ± 2.37^{a} | 29.81 ± 2.48^{ab} | 28.35 ± 3.99^{abc} | 29.73 ± 3.35^{abcd} |
| experiment | | | | |
| State one | | | | |
| 1^{st} to 20^{th} day | 29.76 ± 2.10^{a} | 27.44 ± 2.63^{B} | 28.94 ± 2.28^{BC} | 28.74 ± 1.96^{BCD} |
| 21^{st} to 37^{th} day | 30.37 ± 2.55^{a} | 30.93 ± 3.40^{ab} | 29.85 ± 3.17^{abc} | 29.12 ± 2.28^{abcd} |
| Average: 1^{st} to 37^{th} | 30.07 ± 3.11^{a} | 29.19 ± 3.30^{ab} | 29.70 ± 2.17^{abc} | 29.01 ± 2.17^{abcd} |
| day | | | | |
| Stage two | 27.75 ± 2.30^{a} | 26.72 ± 1.23^{ab} | 26.37 ± 3.32^{abc} | 25.41 ± 1.42^{cd} |

| | | |
|---|---|---|
| Stage three | 27.31 ± 1.25^{a} | 29.08 ± 2.07^{ab} |

(Note:
In the table, same letters in a row means that the difference of their values is insignificant, i.e. p > 0.05, different letters in a same row means that the difference of their values is significant p < 0.05; different letter size in a column means their values are significant, i.e. p < 0.01)

Table 21 above shows that the 3.5 wt % FCM yield of the Group I of cows increased by 3.55% (=[29.76−28.74]/28.74×100%) during 1^{st} to 20^{th} day of the experiment and by 4.29% (=[30.37−29.12]/29.12×100%) during the 21^{st} to 37^{th} day of the experiment as compared to that of the control group of cows. This illustrates that the composition is also effective in increasing the 3.5% FCM yield.

In stage two of the experiment, although the three test groups of cows were stopped feeding on the cysteamine-containing diet, their 3.5 wt % FCM yield was higher than the control group of cows that was feeding on the cysteamine-containing diet. This illustrates that the effect of cysteamine persists for a while after administration thereof was stopped and that it takes some time for cysteamine to take effect in the physiology of the animals.

TABLE 22
Effect of the composition on milk protein content
(wt %)

| | Group | | | |
|---|---|---|---|---|
| | | | | Control |
| Time | Test Group I | Test Group II | Test Group III | Group |
| Before | 2.63 ± 0.24^{a} | 2.81 ± 0.18^{ab} | 2.78 ± 0.20^{abc} | 2.67 ± |
| experiment | | | | 0.29^{abcd} |
| State one | | | | |
| 1^{st} to 20^{th} day | 2.73 ± 0.21^{a} | 2.85 ± 0.20^{ab} | 2.85 ± 0.23^{abc} | 2.81 ± |
| | | | | 0.19^{abcd} |
| 21^{st} to 37^{th} day | 2.70 ± 0.22^{a} | 2.89 ± 0.14^{ab} | 2.80 ± 0.22^{abc} | 2.83 ± |
| | | | | 0.10^{abcd} |
| Average: 1^{st} to | 2.72 ± 0.21^{a} | 2.87 ± 0.17^{b} | 2.83 ± 0.22^{abc} | 2.82 ± |
| 37^{th} day | | | | 0.14^{abcd} |
| Stage two | 2.65 ± 0.17^{a} | 2.84 ± 0.09^{b} | 2.72 ± 0.19^{ab} | 2.76 ± |
| | | | | 0.10^{ac} |

| | | |
|---|---|---|
| Stage three | 2.73 ± 0.18^{a} | 2.89 ± 0.11^{b} |

(Note:
In the table, same letters in a row means that the difference of their values is insignificant, i.e. p > 0.05, different letters in a same row means that the difference of their values is significant p < 0.05; different letter size in a column means their values are significant, i.e. p < 0.01)

TABLE 23
Effect of the composition on milk protein yield
(g/cow/day)

| | Group | | | |
|---|---|---|---|---|
| Time | Test Group I | Test Group II | Test Group III | Control Group |
| Before | 696.88 ± 63.64^{a} | 754.59 ± 49.45^{ab} | 744.93 ± 52.86^{abc} | 713.29 ± 76.97^{abcd} |
| experiment | | | | |
| State one | | | | |
| 1^{st} to 20^{th} day | 797.97 ± 61.72^{a} | 766.37 ± 52.56^{ab} | 757.05 ± 61.33^{abc} | 789.33 ± 52.52^{abcd} |
| 21^{st} to 37^{th} day | 731.09 ± 58.21^{a} | 786.03 ± 39.39^{b} | 736.74 ± 56.89^{ac} | 744.99 ± 25.83^{acd} |
| Average: 1^{st} to | 764.53 ± 58.24^{a} | 776.15 ± 45.16^{ab} | 746.90 ± 59.58^{abc} | 767.16 ± 40.93^{abcd} |
| 37^{th} day | | | | |
| Stage two | 714.85 ± 46.94^{a} | 737.67 ± 23.49^{ab} | 692.29 ± 47.25^{ac} | 691.23 ± 23.95^{acD} |

| | | |
|---|---|---|
| Stage three | 696.00 ± 45.47^{a} | 781.20 ± 30.82^{b} |

(Note:
In the table, same letters in a row means that the difference of their values is insignificant, i.e. p > 0.05, different letters in a same row means that the difference of their values is significant p < 0.05; different letter size in a column means their values are significant, i.e. p < 0.01)

Table 23 above similarly shows the effect of the composition although the milk protein yield is expressed in gram per cow per day.

Table 23 shows that when the control group of cows was administered with 40 g of cysteamine per cow every other day, the milk protein content increased by 5.86% (=[2.89 wt %−2.73 wt %]/2.73 wt %×100%). This illustrates that administering cysteamine at a relatively high dose every other day can be more effective in increasing the milk protein yield than administering cysteamine at a relatively low dose daily.

TABLE 24
Number of somatic cells in milk produced by the
cows (×10³/ml)

| | Group | | | |
|---|---|---|---|---|
| Time | Test Group I | Test Group II | Test Group III | Control Group |
| Before | 1097.4 ± 1070.2^{a} | 803 ± 1113.4^{ab} | 126.9 ± 87.1^{abc} | 99.9 ± 136.5^{abcd} |
| experiment | | | | |
| State one | | | | |
| 1^{st} to 20^{th} day | 296.25 ± 452.33^{a} | 727.63 ± 917.02^{ab} | 395.50 ± 643.44^{abc} | 129.63 ± 152.56^{acd} |
| 21^{st} to 37^{th} day | 360.25 ± 479.59^{a} | 613.38 ± 556.70^{ab} | 220.00 ± 348.65^{abc} | 525.5 ± 917.59^{abcd} |
| Average: 1^{st} to | 328.25 ± 451.56^{a} | 670.50 ± 735.22^{ab} | 307.75 ± 508.08^{ac} | 327.56 ± 667.51^{acd} |
| 37^{th} day | | | | |
| Stage two | 322.9 ± 413.5^{a} | 385.00 ± 304.9^{ab} | 187.1 ± 203.6^{abc} | 224.4 ± 319.2^{abcd} |

| | | |
|---|---|---|
| Stage three | 399.30 ± 560.40^{a} | 335.50 ± 334.00^{ab} |

(Note:
In the table, same letters in a row means that the difference of their values is insignificant, i.e. p > 0.05, different letters in a same row means that the difference of their values is significant p < 0.05; different letter size in a column means their values are significant, i.e. p < 0.01)

Table 24 above shows that, in the test Group I of cows, the number of somatic cells in milk decreased significantly. Similar decrease is observed in the milk produced by the test Group II of cows. The number of somatic cells in milk produced by control group of cows however increased. Since the number of somatic cells in milk is generally indicative of the overall health of the animals, the above data illustrates that the overall health of dairy animals administered with cysteamine will improve.

The above experimental results illustrate that cysteamine or a cysteamine composition is effective for improving lactation of lactating animals.

The contents of each of the references discussed above, including the references cited therein, are herein incorporated by reference in their entirety. It is to be noted that numerous variations, modifications, and further embodiments are possible and accordingly, all such variations, modifications and embodiments are to be regarded as being within the scope of the present invention.

1. A method for improving lactation of lactating animals comprising administering to said animals an effective amount of cysteamine, a salt thereof or a composition containing cysteamine or a salt thereof.

2. The method according to claim 1 wherein said improving lactation is an increase in milk yields.

3. The method according to claim 1 wherein said improving lactation is an increase in fat-corrected milk yields.

4. The method according to claim 1 wherein said improving lactation is an increase in mild fat content therein.

5. The method according to claim 1 wherein said improving lactation is an increase in milk protein content therein.

6. The method according to claim 1 wherein said composition comprises substantially 1 to 95 wt % cysteamine having the chemical formula of NH₂—CH₂—CH₂—SH, or a salt thereof.

7. The method according to claim 6 wherein said cysteamine-containing composition comprises substantially 30 wt % cysteamine.

8. The method according to claim 1 wherein said composition comprises substantially 1 to 80 wt % of a stabilizer.

9. The method according to claim 8 wherein said stabilizer is selected from the group consisting of cyclodextrin or a derivative thereof.

10. The method according to claim 8 wherein said composition comprises substantially 10 wt % of said stabilizer.

11. The method according to claim 1 wherein said composition further comprises ingredient(s) selected from the group consisting of a bulking agent, a disintegration agent and a coated carrier.

12. The method according to claim 11 wherein said coated carrier is a solid carrier.

13. The method according to claim 11 wherein said coated carrier is has a coating soluble in intestines of said animals.

14. The method according to claim 11 wherein said coated carrier exhibits a multi-layer structure in said composition.

15. The method according to claim 11 wherein said coated carrier is adapted to remain un-dissolved at a pH of from about 1.5 to 3.5.

16. The method according to claim 1 wherein said lactating animals are dairy cows.

17. A feed for improving lactation of lactating animals comprising a composition containing cysteamine or a salt thereof and a stabilizer.

18. The feed according to claim 17 wherein said improving lactation is an increase in milk yields.

19. The feed according to claim 17 wherein said improving lactation is an increase in fat-corrected milk yields.

20. The feed according to claim 17 wherein said improving lactation is an increase in milk fat content therein.

21. The feed according to claim 17 wherein said improving lactation is an increase in mild protein content therein.

22. The feed according to claim 17 wherein said composition comprises substantially 1 to 95 wt % cysteamine having the chemical formula of NH₂—CH₂—CH₂—SH, or a salt thereof.

23. The feed according to claim 22 wherein said cysteamine-containing composition comprises substantially 30 wt % cysteamine.

24. The feed according to claim 17 wherein said stabilizer is selected from the group consisting of cyclodextrin or a derivative thereof.

25. The feed according to claim 17 wherein said composition comprises substantially 1 to 80 wt % of said stabilizer.

26. The feed according to claim 17 wherein said composition further comprises ingredient(s) selected from the group consisting of a bulking agent, a disintegration agent and a coated carrier.

27. The feed according to claim 26 wherein said coated carrier is a solid carrier.

28. The feed according to claim 26 wherein said coated carrier has a coating soluble in intestines of said animals.

29. The feed according to claim 26 wherein said coated carrier exhibits a multi-layer structure in said composition.

30. The feed according to claim 26 wherein said coated carrier is adapted to remain undissolved at a pH of from about 1.5 to 3.5.

31. A The feed according to claim 17 wherein said lactating animals are dairy cows.

32. The feed according to claim 17 wherein the feed comprises substantially 400 to 1000 ppm of said composition.

33. The feed according to claim 17 wherein in its dry state, the feed comprising comprises substantially 1000 to 2174 ppm of said composition.

34. The feed according to claim 17 wherein the feed comprises substantially 120 to 300 ppm of cysteamine.

35. The feed according to wherein the feed in its dry state comprises substantially 200 to 650 ppm of cysteamine.

36. The feed according to further comprising other foodstuffs selected from the group consisting of normal premix, cornmeal, cotton seed, wheat gluten, maize silage rutabaga, sugar beet pulp, apple pulp, ryegrass, fescue grass, alfalfa, feed concentrate and feed supplement.

37. A method for improving lactation of lactating animals comprising:
(a) producing a final feed by mixing a composition containing cysteamine or a salt thereof and a stabilizer with a suitable basal feed for said animals; and
(b) feeding said animals with said final feed.

38. The method according to claim 37 wherein said mixing in said step (a) comprises directly mixing said composition with said basal feed.

39. The method according to claim 37 wherein said mixing in said step (a) comprises firstly preparing a premix including cysteamine or said composition, and subsequently mixing said pre-mix with said basal feed.

40. The method according to claim 39 wherein said premix is prepared by mixing cysteamine or said composition with a food material selected from a the group including consisting of cornmeal.

41. The method according to claim 39 wherein said premix comprises 5 to 25 wt % of said composition.

42. The method according to claim 39 wherein said premix comprises 10 to 20 wt % of said composition.

43. The method according to claim 37 further comprising feeding said animals with substantially 5.64 to 12.71 g cysteamine, or a salt thereof, per animal per day.

44. The method according to claim 37 further comprising feeding said animals with substantially 18.79 to 42.36 of said composition per animal per day.
